(19) United States
(12) Patent Application Publication
(10) Pub. No.: US 2005/0227942 A1
(43) Pub. Date: Oct. 13, 2005

(54) Method and agent for the prevention, inhibition and treatment of sepsis

(76) Inventor: Andreas Bergmann, Berlin (DE)

Correspondence Address: MAYER, BROWN, ROWE & MAW LLP, 1909 K STREET, N.W., WASHINGTON, DC, 20006, US

(21) Appl. No.: 10/516,619

(22) PCT Filed: Apr. 2, 2003
(86) PCT No.: PCT/EP03/03448

§ 371 Date: May 25, 2005

(30) Foreign Application Priority Data
Jun. 4, 2002 (EP) 02012515.9

(51) Int. Cl. A61K 31/739; A61K 39/00

(52) U.S. Cl. 514/54; 424/140.1

(57) ABSTRACT

The invention relates to the use of Asialo-Gₘ₁ (Agₘ₁) and/or GM gangliosides, and substances which simulate the carbohydrate part of said gangliosides in terms of binding to anti-AGₘ₁ antibodies and/or anti-Gₘ₁ antibodies, for producing an agent which blocks or binds to anti-AGₘ₁ antibodies and/or anti-Gₘ₁ antibodies which bind to natural killer cells (NKC), said agent being administered to patients at high risk of sepsis and to patients with sepsis, in whom such antibodies have been detected, for the prevention, inhibition and treatment of sepsis.

The present invention relates to novel methods for sepsis prevention or sepsis treatment and the agents which can be used in such methods. It is based on a novel diagnostic finding, namely that antibodies (autoantibodies) which have the so-called properties of inhibiting “natural killer cells” (NK cells) were found with high sensitivity in the sera of patients suffering from sepsis, and on the preventive and therapeutic measures which the Applicant has derived from the critical role which these antibodies can play in the development of the sepsis owing to their NK cell-inhibiting properties.

In particular, the present invention relates to methods for the prevention and treatment of a septic reaction in human patients (patients at risk of sepsis), in which, after a medical intervention and/or a trauma (accident, burn, war injuries, decubitus and the like), a sepsis has developed or could still develop, and, over and above this, also for avoiding a potential risk of a septic reaction in patients who have to undergo, for example, a surgical operation in which a sepsis must be feared as a dangerous complication, for example in the field of visceral surgery, transplantation medicine and high-dose chemotherapy in haematology/oncology.

The term “sepsis” is now used in close association with the term “inflammation”. Inflammations are defined very generally as certain physiological reactions of an organism to different types of external effects, such as, for example injuries, burns, allergens, infections by microorganisms, such as bacteria, fungi and viruses, to foreign tissues which trigger rejection reactions, or to certain endogenous states of the body which trigger inflammation, for example in autoimmune diseases and cancer. Inflammations may occur as harmless, localized reactions of the body but are also typical features of numerous serious chronic and acute diseases of individual tissues, organs, organ parts and tissue parts.

Local inflammations are generally part of the healthy immune response of the body to harmful effects, and hence part of the life-preserving defence mechanism of the organism. However, if inflammations are part of a misdirected response of the body to certain endogenous processes, such as, for example, in autoimmune diseases, and/or are of a chronic nature, or if they reach systemic extents, as in the case of systemic inflammatory response syndrome (SIRS) or in a severe sepsis caused by infection, the physiological processes typical of inflammatory reactions go out of control and become the actual, frequently life-threatening pathological process. Regarding the modern definition of sepsis, reference may be made, for example, to the discussion of the definition of sepsis in K. Reinhart et al., “Sepsis und septischer Schock” [Sepsis and septic shock], in: Intensivmedizin, Georg Thieme Verlag, Stuttgart, New York, 2002, 756-760. According to this, “the clinical picture of sepsis is a separate pathophysiological and clinical entity which, also in terms of treatment, must be described and treated separately from the underlying infection”.

It is now known that the origin and the course of inflammatory processes are controlled by a cascade of substances which are predominantly of a protein or peptide nature or are accompanied by the occurrence of certain biomolecules for a more or less limited time. The endogenous substances involved in inflammatory reactions include in particular those which can be assigned to the cytokines, mediators, vasoactive substances, acute phase proteins and/or hormonal regulators. The inflammatory reaction is thus a complex physiological reaction in which both endogenous substances activating the inflammatory process (e.g. TNF-α, interleukin-1) and deactivating substances (e.g. interleukin-10) are involved.

In systemic inflammations, as in the case of sepsis or of septic shock, the inflammation-specific reaction cascades spread in an uncontrolled manner over the whole body and become life-threatening in the context of an immune response which is excessive or causes dysfunction.

Whereas at least in Europe the systemic bacterial infection detectable by a positive blood culture long characterized the term sepsis, sepsis is now primarily understood as being systemic inflammation which is caused by infection but, as a pathological process, has considerable similarities with systemic inflammations which are triggered by other causes. Thus, the direct detection of bacterial pathogens was recently replaced or supplemented by complex monitoring of physiological parameters and also by the detection of certain endogenous substances shown to be involved in the sepsis process or in the inflammatory process, i.e. specific “biomarkers”.

An introduced endogenous substance which is particularly suitable as a sepsis biomarker is procalcitonin. The determination of procalcitonin as a sepsis marker is the subject of the publication by M. Assicot et al., “High serum procalcitonin concentrations in patients with sepsis and infection”, The Lancet, Vol. 341, No. 8844, 1993, 515-518; and the patents DE 42 27 454 C2 and EP 0 656 121 B1 and U.S. Pat. No. 5,639,617.

The availability of the sepsis marker procalcitonin has given considerable impetus to recent sepsis research, and intensive efforts are now being made to find further biomarkers which can supplement the procalcitonin determination and/or are capable of providing additional information for purposes of fine diagnosis or differential diagnosis. Results of these efforts are to be found in numerous patent applications of the Applicant, in particular in DE 198 47 690 A1 or WO 00/22439, and in a number of still unpublished German Patent Applications (DE 101 19 804.3 or PCT/EP02/04219; DE 101 31 922.3; DE 101 30 985.6) or European Patent Applications (EP 01128848.7; EP 01128849.5; EP 01128850.3; EP 01128851.1; EP 01128852.9; EP 01129121.8; EP 02008840.7 and EP 02008841.5). Reference is hereby made to the content of said patents and patent applications for supplementing the present description.

Since the endogenous substances formed during inflammations are part of the complex reaction cascade of the body, not only are such substances of diagnostic interest but attempts are also currently being made, with considerable effort, to intervene therapeutically in the inflammatory process by influencing the origin and/or the concentration of individual substances of this type, in order to stop at as early a stage as possible the systemic spread of the inflammation which is observed, for example, in sepsis. In this context, endogenous substances which can be shown to be involved in the inflammatory process are also to be regarded as potential therapeutic targets. Attempts starting from certain mediators of the inflammatory process to influence this therapeutically in a positive manner are described, for example, in E. A. Panacek, “Anti-TNF strategies”, Journal für Anästhesie und Intensivbehandlung; No. 2, 2001, 4-5; T. Calandra et al., “Protection from septic shock by neutralization of macrophage migration inhibitory factor”, Nature Medicine, Vol. 6, No. 2, 2000, 164-170; or K. Garber, “Protein C may be sepsis solution”, Nature Biotechnology, Vol. 18, 2000, 917-918. In view of the fairly disappointing results of such therapeutic approaches to date, there is considerable interest in identifying further endogenous biomolecules which are as inflammation- or sepsis-specific as possible and, as therapeutic targets, also open up new prospects for success for the prevention and treatment of sepsis.

A general object of the present invention can therefore be defined as providing novel measures (methods, agents, uses) for the prevention and treatment of sepsis, which arise from the discovery of biomolecules which occur with high sensitivity in sepsis and play a key role in the development and in the course of sepsis.

This object is achieved by uses according to claims 1 to 3 and embodiments thereof according to claims 4 and 5 and agents according to claim 6.

The present invention is based in general on the surprising experimental finding that a certain antibody or autoantibody type known per se in other contexts is found with high sensitivity and at significantly increased levels with extremely high frequency in sera of patients who had subsequently developed a sepsis or already had a sepsis, whereas the same antibody is not detectable or detectable only in substantially smaller amounts in healthy normal persons.

The fact that the antibodies found are those which can eliminate the function of “natural killer cells” (NK cells; cytotoxic lymphocytes) and that it has moreover been found that related antibodies determined in other scientific contexts cross-react with these antibodies which damage the NK cells plays a decisive role regarding the preventive and therapeutic measures described in this Application.

If such antibodies which inhibit NK cells are found at increased levels in virtually all patients suffering from sepsis, they are therefore a therapeutic target for the prevention of sepsis and treatment of sepsis.

In the context of the present invention, as explained in more detail below, they can become the target of preventive, inhibitory and/or therapeutic measures with the use of measures, some of which are basically known per se, or agents according to the invention for fighting and binding pathogenic (auto)antibodies and rendering them harmless and/or of measures with the aim of specifically influencing the antibody formation by the immune system. Furthermore, their detection prior to a sepsis risk event may be a reason for taking greater safety measures, for example in the context of a preventive treatment with antibiotics, for avoiding development of a sepsis.

More precisely, the present invention starts from the surprising results of measurements by the Applicant on sera of normal persons and patients suffering from sepsis with the aid of a ligand binding assay with high sensitivity for antibodies binding to the gangliosides AG_{M1} and G_{M1}. The surprising result was that, in such measurements, antibodies binding to asialo-G_{M1} (anti-AG_{M1} antibodies) and/or antibodies cross-reacting therewith, in particular antibodies binding to monosialo-G_{M1} (anti-G_{M1} antibodies) of the IgG and/or IgA type were found substantially in all measured sepsis sera.

The increased presence of antibodies which bind to asialo-G_{M1} and hence have properties which inhibit or damage NK cells in virtually all sera of patients suffering from sepsis indicates that such antibodies are directly involved in the development of a sepsis and/or can be correlated with an increased risk to a patient at risk of sepsis, i.e. with the risk that the patient will develop a sepsis or with its individual disposition to sepsis.

The physiological binding partners of said antibodies are gangliosides. Gangliosides are glycolipids which are constituents of the extracellular side of the plasma membrane of animal cells and as such also occur in nerve tissue. They contain several monosaccharide units per mole but have no phosphorus content and are assigned to the sphingolipids. Compared with proteins, they tend to be low molecular weight biomolecules. The gangliosides to which the antibodies discussed in the context of the present invention bind are the monosialo-ganglioside referred to generally as G_{M1} and in particular the associated “asialo” compound AG_{M1}. G_{M1} has a polysaccharide chain of 4 sugar monomer units which comprise two D-galactose units, one N-acetylgalactosamine unit and one D-glucose unit, the latter being bound to a so-called ceramide moiety. In the ganglioside G_{M1}, an N-acetylneuraminic acid radical (NANA; sialic acid or o-sialinic acid radical; “monosialo” radical), which is missing in the sialinic acid-free asialo-G_{M1} (AG_{M1}), is bound to the D-galactose unit arranged inside the polysaccharide chain.

Said gangliosides and related compounds are associated with numerous important biological functions of the human body, including, for example, axonal growth and neuronal differentiation, receptor functions and participations in various immune reactions of the body and in signal transduction and cell-cell recognition.

It has long been known that antibodies or autoantibodies which bind to the ganglioside G_{M1}, in particular its carbohydrate structures, and to carbohydrate structures of other molecules which resemble these (“simulate these”) can be detected in the human body in certain contexts. The physiological role of such antibodies and their possible importance for clinical diagnosis are the subject of numerous scientific investigations. However, the detection of anti-G_{M1} antibodies has not to date been correlated with disadvantageous physiological reactions which are important for the development of sepsis and which may be attributable to properties, known per se, of antibodies binding to asialo-G_{M1}.

By far the predominant part of all published papers are concerned with the role and the diagnostic significance of anti-ganglioside antibodies in neuropathies, for example in immunomediated motor neuropathies, such as Guillain-Barré syndrome (radiculoneuritis, polyradiculitis) and the related (Miller-)Fisher syndrome. An increased occurrence of anti-G_{M1} autoantibodies in some patients was also reported in association with Alzheimer's disease. Furthermore, they were found in individual HIV patients. That they also occur with very high sensitivity in cancer is a still unpublished novel finding of the Applicant, on which the inventions disclosed in the European Patent Applications EP 02009884.4 and EP 02009882.8 are based. Reference is hereby made to the two last-mentioned European Patent Applications and the list of references for EP 02009884.4 for supplementing the statements in the present Application.

The findings which form the basis of the inventions in the present Application, and the preventive and therapeutic measures derived therefrom, are explained in more detail below.

In order to avoid unjustifiably narrow and restrictive interpretations of the terms used in the present Application and the associated claims, some of the most important terms are to be defined in particular below for the purposes of the present Application:
“Antibody”: This term includes, without distinguishing between different methods of genesis and formation, antibodies both against external antigens and against endogenous structures, i.e. autoantibodies, where the latter may also have become autoantibodies by antigen cross-reactions from antibodies against external antigens and may have preserved their binding capability with respect to external antigens.
When, for example, it is stated that an antibody binds “to ganglioside structures and to antigen structures simulating ganglioside structures” or is “reactive towards gangliosides or certain gangliosides”, where reactive means “reactive in the context of specific binding”, it should be sufficiently defined by this definition without, for example, its specific binding also to additional other antigen structures, or its practical determination using reagents (for immobilization or marking or as competitors) with molecular structures which only simulate AGm₁, in particular the carbohydrate structure thereof, playing a role for the definition as antibodies according to the invention.
“Ganglioside” In the context of the present invention, the term “ganglioside” primarily represents the gangliosides AGm₁ in the characterization of the binding behaviour of the antibodies to be determined. However, the term is also intended to include related gangliosides not investigated to date, if it is found that antibodies also binding to these gangliosides and having a comparable diagnostic significance are found in sepsis sera.
“Simulation” When, in the context of the present Application, it is stated that, in addition to the specific gangliosides (AG_{M1} and G_{M1}), substances or compounds “simulating” binding properties and effects thereof may also be used, what is meant by this is that there are compounds which have carbohydrate structures (including bacterial toxins) and which bind to the antibodies in question like said gangliosides. Such compounds can therefore be potentially used, like the gangliosides themselves, for specific binding of the antibodies (for example for the purpose of removing them from the blood circulation) or for their blocking (and hence rendering them harmless).
   In the stated context, substances “simulating” the binding behaviour of gangliosides can be found, for example, with the aid of a screening method in which a biological sample (in particular a serum) which has a high anti-ganglioside antibody titre and whose binding behaviour was determined in a given assay is brought into contact with a candidate substance to be tested and the antibody binding is determined again in the same assay. A substantial reduction or elimination of the antibody binding in the assay is an indication that the substance investigated is a “ganglioside-simulating substance”. This test can also be carried out in association with establishing a patent infringement by agents which correspond to those specifically disclosed or claimed in the present Application.

Further meanings of the terms are evident to a person skilled in the art from the introductory and following description of the invention and its embodiments and the literature cited therein.

In the description below, for explaining the diagnostic findings which are the basis of the preventive and therapeutically useful teachings, reference is made to figures which show the following:

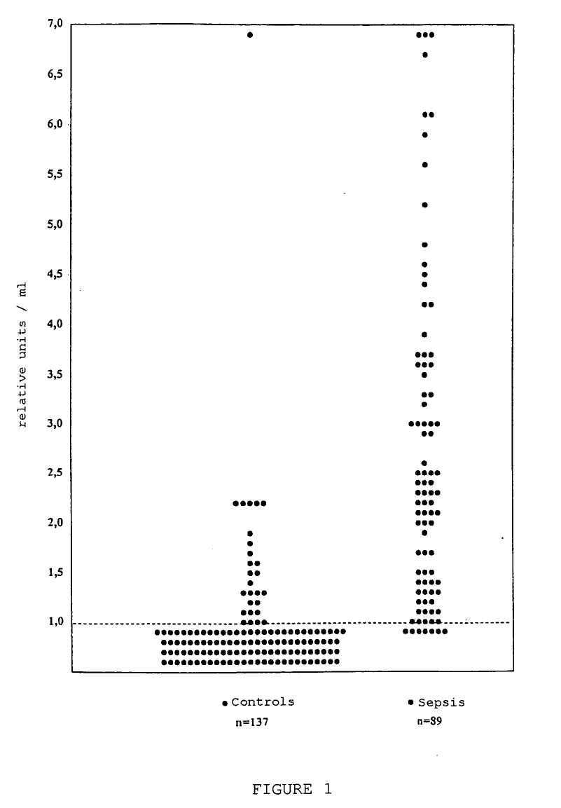

FIG. 1 shows a graph of the results of the measurement of antibodies of the IgG class which bind to monosialo-G_{M1}, in sera of 137 control persons, compared with the results of the measurement of 89 sera of patients suffering from sepsis;

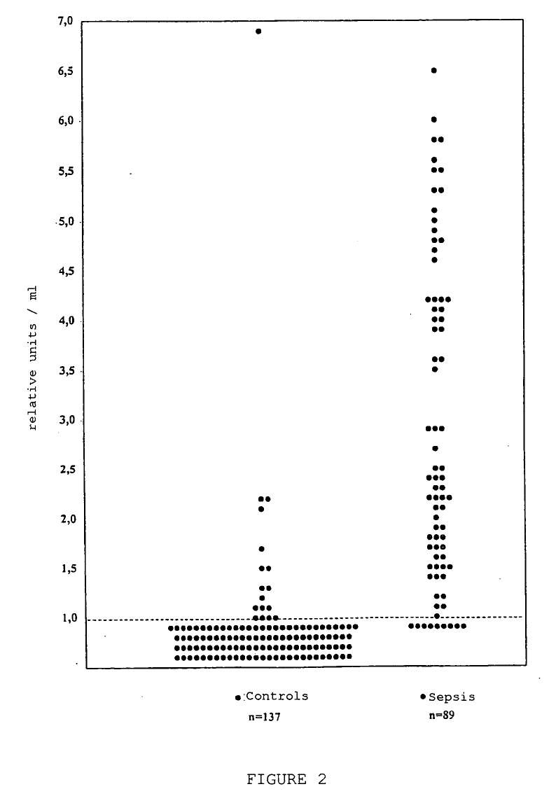

FIG. 2 shows the results of a measurement of the same sera as in FIG. 1 for antibodies of the IgA class which bind to monosialo-G_{M1};

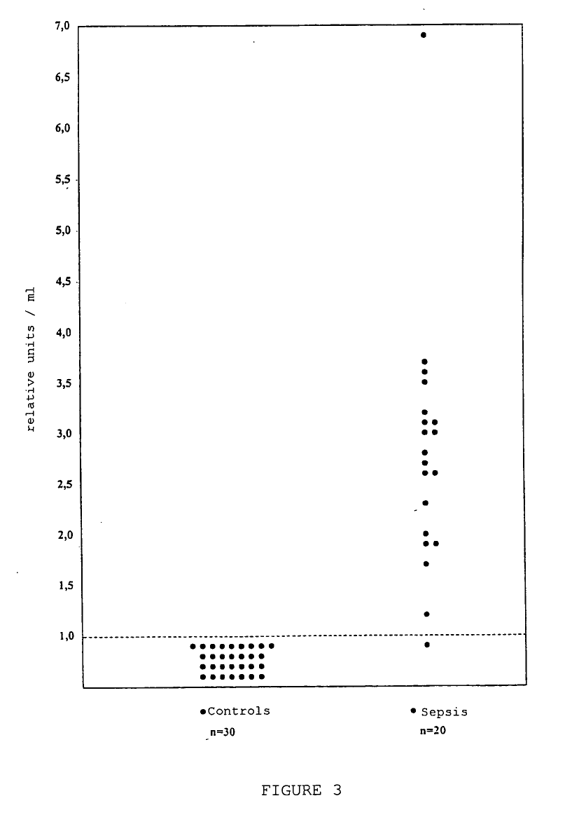

FIG. 3 shows the results of the determination of antibodies of the IgG class which bind to asialo-G_{M1}, in sera of 30 normal persons (controls), compared with the results of the measurement of 20 sera of patients suffering from sepsis (all sera are partial groups of the sera measured in FIGS. 1 and 2);

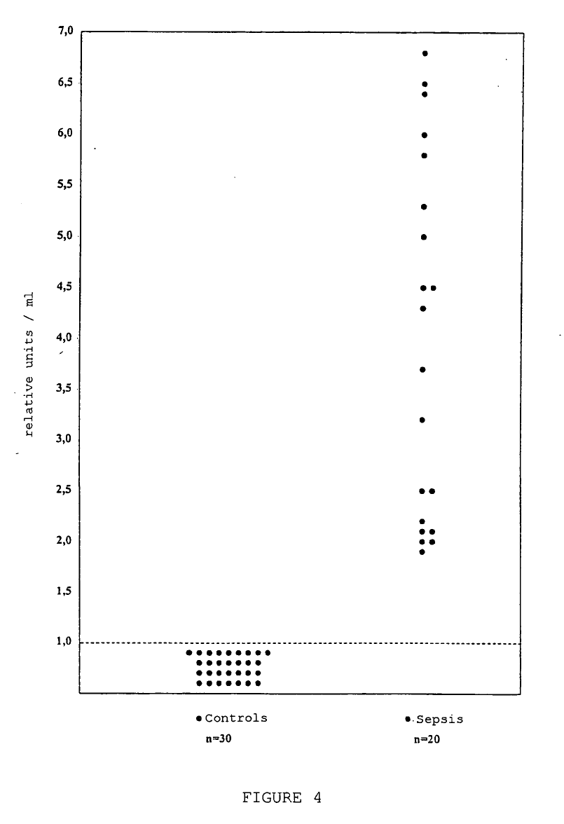

FIG. 4 shows the results of the determination of antibodies of the IgA class which bind to asialo-G_{M1}, in the same sera as in FIG. 3.

The invention is based on measurements of sera carried out by the Applicant, substantially only the principle of measurement and the results obtained being reproduced in the present Application. A detailed disclosure of the measurements carried out is to be found in the prior unpublished European Patent Application 02009884.4 of the Applicant and a further application of the Applicant filed simultaneously with the present Application. Reference is made to both applications for supplementing the disclosure of the present Application:
1. Determination of (auto)antibodies of the IgG and IgA type which bind to the gangliosides AG_{M1} and G_{M1} in sera of healthy normal persons (controls) and patients suffering from sepsis.

Using test tubes coated with gangliosides (AG_{M1} and G_{M1}) (GA-CTs), the free binding sites of which had been saturated with BSA, series measurements were carried out on control sera and test sera. As described in more detail in the prior unpublished Application of the Applicant EP 02009884.4, and a further European Patent Application of the Applicant filed simultaneously with the present Application, antibodies from the respective serum (control serum and test serum) were bound, in a first incubation step, to the gangliosides used for the coating, and then, for separate determination of antibodies of the IgG and IgA type, the bound antibodies were detected using marked animal (goat) anti-human IgG or anti-human IgA immunoglobulins. The bound marked immunoglobulins were detected and quantified (in relative units, based on serum having the highest concentration of the respective antibody to be determined) on the basis of their marking (acridinium ester as chemiluminescence marker).

In order to obtain a measured signal representative of the amount of antibody, it is necessary to subtract a background signal which was determined under identical measuring conditions for the respective identical serum using those test tubes which, apart from the lacking ganglioside coating, were identical to the test tubes used for the actual measurements.

137 control sera (blood donor sera and—for avoiding age-related influences on the antibody concentrations—sera of normal persons of different ages from old people's homes and of the Applicant's employees) served as control sera for the antibody assays using GA-CTs which were coated with G_{M1}. For the antibody assays using GA-CTs which were coated with AG_{M1}, a partial group of these sera which comprised only 30 sera was measured.

89 sera of patients suffering from sepsis served as test sera for the antibody assays using GA-CTs which were coated with G_{M1}. Exact clinical documentation existed for each test serum. For the antibody assays using GA-CTs which were coated with AG_{M1}, a partial group of these sera which comprised only 20 of the sera of the patients suffering from sepsis was measured.

The results of the determinations of antibodies of the IgG and IgA classes using GA-CTs which were coated with G_{M1} are shown by way of example in FIGS. 1 and 2.

The corresponding results obtained using GA-CTs which were coated with AG_{M1} are shown in FIGS. 3 and 4.

It should be expressly pointed out that numerous sera in which significantly increased anti-AG_{M1}, or anti-G_{M1} antibody titres were found originated from patients from whom the blood sample had been taken shortly (about 2 h) after the sepsis risk event and who developed the typical sepsis symptoms only later on.

It should furthermore be pointed out that, in an experiment also to determine the corresponding antibodies of the IgM type analogously to the determinations of the antibodies of the IgG and IgA type, no levels for such antibodies of the IgM type which were increased to a diagnostically relevant extent were found in the sepsis sera (results not shown).
2. Discussion of the findings and measures according to the invention for sepsis prevention and treatment

As impressively shown by the measured results summarized in FIGS. 1 to 4, the determination of antibodies of the classes IgA and/or IgG which bind to gangliosides (AG_{M1} and/or G_{M1}) permits a clear distinction of the control group from the patients suffering from sepsis, by virtue of the fact that substantially increased AG_{M1} and G_{M1} antibody titres are found in virtually all (FIG. 1: 82 out of 89, i.e. 92%; FIG. 2: 80 out of 89, 90%; FIG. 3: 19 out of 20, 95%; FIG. 4: all 20, 100%) of the sepsis sera investigated.

The detection of substantially increased concentrations of antibodies of the IgA and IgG type also in patient's serum samples which had been obtained only a short time (about 2 h) after the “sepsis risk event” (e.g. operation, accident, burn), and the lack of evidence of antibodies of the IgM type, ruled out the possibility that the detected antibodies were formed only as a result of the “sepsis risk event” or of a bacterial infection associated therewith. However, this means that either the antibodies were already present beforehand in the respective patient suffering from sepsis and/or that the activation of the presensitized immune system of the patient in the manner of a “booster” effect, triggered by the sepsis risk event, led to intensive antibody production.

The fact that the AG_{M1} or G_{M1} antibodies were found at substantially increased levels in substantially all measured sepsis sera (90 to 100%) is therefore to be interpreted so as to mean that the formation of a sepsis is either due directly to the presence of the antibodies in question in the respective patient or is at least the consequence of activation of the “molecular machinery” (in the form of B-cells) already present in the patient owing to prior immunization, which begins its intensive antibody production under the influence of the “sepsis risk event” or of an infection associated therewith. Patients without these antibodies or the presensitization required for their rapid production probably do not develop a sepsis or develop one only with difficulty, based on the experimental results to date.

The observed results can be explained: it is known that the so-called “natural killer cells” (NK-cells; cytotoxically active lymphocytes) have, on their surface, asialo-G_{M1} structures to which anti-AG_{M1}, antibodies can specifically bind and thus deactivate and destroy the NK-cells. This is even routinely used in the field of animal experiments which employs experimental animals in which tumours are to be artificially produced to eliminate the immune defence of the experimental animal by administering anti-AG_{M1}, antibodies in combination with a carcinogen or a tumour nucleus, so that the experimental cancer—desired in the animal model—can develop (Hugh F. Pross et al., Role of Natural Killer Cells in Cancer, Nat Immun 1993; 12:279-292; Lewis L. Lanier et al., Arousal and inhibition of human NK Cells, Immunological Reviews 1997, Vol. 155:145-154; Yoichi Fuji et al., IgG Antibodies to AsialoGM1 Are More Sensitive than IgM Antibodies to Kill in vivo Natural Killer Cells and Prematured Cytotoxic T Lymphocytes of Mouse Spleen, Microbiol. Immunol. Vol. 34(6), 533-542, 1990; N. Saijo et al., Analysis of Metastatic Spread and Growth of Tumor Cells in Mice with Depressed Natural Killer Activity by Anti-asialo GM1 Antibody or Anticancer Agents, J Cancer Res Clin Oncol (1984) 107: 157-163; Sonoku HABU et al., Role of Natural Killer Cells against Tumor growth in Nude Mice—A Brief Review, Tokai J Exp Clin Med., Vol. 8, No. 5, 6: 465-468, 1983; Lewis L. Lanier, N K Cell Receptors, Annu. Rev. Immunol. 1998, 16: 359-93; Theresa L. Whiteside et al., The role of natural killer cells in immune surveillance of cancer; Current Opinion in Immunology 1995, 7:704-710; Tuomo Timonen et al, Natural killer cell-target cell interactions, Current Opinion in Cell Biology 1997, 9:667-673).

However, active NK-cells play an extremely important role in the human immune defence, also in the case of sepsis or severe bacterial infections. Thus, for example, Shuiui Seki et al., in: Role of Liver NK Cells and Peritoneal Macrophages in Gamma Interferon and Interleukin-10 Production in Experimental Bacterial Peritonitis in Mice, Infection and Immunity, Vol. 66, No. 11, 1998, 5286-5294, describe the important role of NK-cells for the production of inflammation-promoting and anti-inflammatory cytokines. They show that switching off the NK cells artificially with experimental use of anti-AG_{M1}, antibodies leads to inhibition of the production of the anti-inflammatory interferon-γ. Effects of surgical stress and an endotoxin-induced sepsis on the NK-cell activity have already been investigated, in particular in: P. Toft et al., in: The effect of surgical stress and endotoxin-induced sepsis on the NK-cell activity, distribution and pulmonary clearance of YAC-1 and melanoma cells, APMIS 1999; 107:359-364. A possible influence of physiologically formed antibodies with NK-cell reactivity, however, is not taken into account in any of the papers mentioned.

The scientific literature does not reveal any discovery which might suggest the method according to the invention. It is true that a determination of anti-ganglioside antibodies in connection with severe acute infectious diseases was described in some papers. Such an infectious disease is Chagas disease caused by the parasite Trypanosoma cruzi (cf. D. H. Bronia et al., in: Ganglioside treatment of acute Trypanosoma cruzi infection in mice promotes long-term survival and parasitological cure, Annals of Tropical Medicine & Parasitology, Vol. 93, No. 4, 341-350 (1999) and the literature cited therein). The last-mentioned paper speculates that an observed, substantially advantageous effect of administering exogenous ganglioside to mice infected with the parasite T. cruzi might induce in said mice production of anti-ganglioside antibodies, which then react with glycolipids of the membrane T. cruzi and should thus result in the death of the parasite. Owing to the findings in the present Application, such an explanation is not very probable: owing to their NK cell-inhibiting effect, the anti-ganglioside antibodies observed in the case of Chagas disease are not, as assumed, a healing-promoting factor but instead a disease-inducing or disease-promoting factor. As a result of the administration of exogenous gangliosides, which as such cannot be regarded as antigens, the anti-AG_{M1} antibodies are in fact not formed but probably blocked. Consequently, the effect of the NK cells may be restored in the mice (or in patients), and the immune system can overcome the parasite. All papers with which we are familiar establish no relationship at all between anti-asialo-G_{M1}, antibodies and the origin and worsening of sepsis and therefore can neither anticipate nor suggest the method according to the invention.

This also applies to other papers which show that administration of exogenous gangliosides or certain ganglioside derivatives in experimental animal models has an anti-inflammatory effect or significantly increases the survival rate of mice to which the bacterial toxin LPS had been administered (cf. Silvia G. Correa et al., in: Anti-inflammatory effect of gangliosides in the rat hindpaw edema test, European Journal of Pharmacology, 199 (1991) 93-98; Amico-Roxas M., et al., in: Anti-inflammatory action of AGF₄₄, a ganglioside ester derivative, Drugs Exptl. Clin. Res. XVIII(6) 251-259 (1992); James J. Mond et al., in: Inhibition of LPS-Mediated Cell Activation In Vitro and In Vivo by Gangliosides, Circulatory Shock 44:57-62 (1995). In none of the papers are the observed effects of the interactions of the administered gangliosides with anti-ganglioside antibodies explained. The latter are not even discussed in the given context.

Such an interaction in the context of antibody blocking can conclusively explain the findings described. The conceptual principles of the present invention can be intensified as follows: it is to be assumed that the patients who are suffering from sepsis and whose sera were measured have the specific antibodies or the predisposition for their rapid production (in the context of sensitization) before the “sepsis risk event”. The sensitization of a patient with respect to the production of anti-ganglioside antibodies may have taken place, for example, as a reaction to some exogenous, antigenic stimulus (for example a general infection with Campylobacter jejuni or Heliobacter pylori or, if appropriate, corresponding environmental substances), independently of the subsequent sepsis. It may remain latent for a long time thereafter. However, once the production of anti-asialo-G_{M1} antibodies has been initiated or increases greatly in a human individual, this patient has, as a disposition, the preconditions that there will be damage to the NK cells and hence the immune defence in certain physiological stress situations, such as infections and other events with high NK cell activity (for example cell degeneration through mutagenic events; a sepsis risk situation), by the anti-AG_{M1} antibodies and antibodies cross-reacting therewith. There is an increased risk that a defence reaction which is triggered by, for example, a “sepsis risk stress” and requires intervention by the NK cells will also stimulate the production of the above-mentioned antibodies, and that these will then cancel out the effect of the NK cells. The regulatory cycle of the immune response is then decisively disturbed, and a sepsis may develop.

The detection of naturally occurring anti-AG_{M1} antibodies and anti-ganglioside antibodies cross-reacting therewith, e.g. anti-G_{M1} antibodies, and the increased levels of such antibodies in sera of patients suffering from sepsis therefore mean that such antibodies may represent a previously unconsidered parameter influencing the infection- or inflammation-specific cytokine cascade, in that they intervene in the natural cytokine regulation cycle and, by disturbing or switching off the NK-cells, can cause this to malfunction and trigger a septic reaction in the patient.

It is therefore to be assumed that the anti-AG_{M1} or anti-G_{M1} antibody titres found at significantly increased levels in all sepsis sera which were measured in the above-mentioned assays constitute one of the preconditions for the origin of a sepsis, and the presence of such antibodies has a sepsis-inducing or sepsis-enhancing effect.

The determination of such antibodies is therefore suitable for determining the risk situation (individual disposition) and for the prognosis in a patient who is to be classed as a sepsis risk patient.

A method which comprises determining the presence and/or the amount of anti-AG_{M1} antibodies and antibodies cross-reacting therewith in a biological fluid of a sepsis risk patient, and taking sepsis-preventing measures in the event of their detection, is derived therefrom. Thus, for example, a preventive treatment with antibiotics can be carried out for rapid preventive therapeutic intervention.

An administration of suitable substances for said purposes can also be carried out as a purely routine preventive measure, i.e. without prior determination of the antibodies. However, this is not very advisable in view of the seriousness of an incorrectly estimated risk of sepsis.

Since, without external stress, the antibody titres may in certain circumstances be very low, it is within the scope of the invention to carry out the antibody determination after an in vivo stimulation of the antibody formation of a patient at risk of sepsis, for example before a surgical operation, using safe stimulants. In view of the IgA antibodies found at substantially increased levels (cf. FIGS. 2 and 4), the antibody determination should also be capable of being carried out expressly by suitable assays in body secretions (e.g. saliva, mucous).

The increased titres of anti-ganglioside (auto)antibodies in patients suffering from sepsis are therefore of serious medical importance, namely as a pathogenic factor. With this knowledge, it is possible to develop novel, promising therapeutic approaches:
1. Measures for the intracorporeal blocking of the pathogenic antibodies by administering to the patient agents (which of course must have the required compatibility) which are suitable for saturating (“for blocking”) the antibodies binding to AG_{M1}.

The gangliosides themselves are such agents, and their good compatibility for human patients is known from other contexts in which they have already been administered (they are administered in large amounts to patients suffering from Parkinson's disease). Where such gangliosides are said to have already been administered in relevant contexts for therapeutic purposes to humans without the above-mentioned contexts having been recognized, the agents used are to be excluded from the scope of protection of the present invention.

In addition to the administration of the gangliosides themselves, in dosage forms and amounts suitable for the purpose according to the invention, ganglioside-“simulating” substances can also be administered, as already explained above. One possibility for identifying such substances, in particular those which bind more strongly to the antibodies than the gangliosides themselves, is described further above, in association with the definition of “simulation”.

Candidates for such substances may be, for example, oligosaccharides which consist of the sugar tetramer of the AG_{M1} molecule—without ceramide and sialinic acid radical—or contain these, and derivatives thereof.
2. Removal of the antibodies from the patient's circulation. This can be effected, for example, by extracorporeal binding to solid affinity materials (“plasmapheresis”). For the production of the plasmapheresis affinity columns used as binders, it is also possible, if appropriate, to use high-affinity carbohydrate structures which bind the antibodies with higher affinity than the gangliosides themselves and, owing to their toxin properties, cannot be administered to a patient. Regarding the question of the production of T-cell anergy, reference may additionally be made to the content of EP 705 107 A1 and the further references mentioned therein.
3. Reduction of the endogenous production of the specific antibodies by administration of agents for blocking antigen-presenting cells or for producing T-cell anergy. These measures may be accompanying effects of the measures mentioned under 1., but may advantageously also be applied in a targeted manner by choosing other administration routes (e.g. oral) or amounts which are suitable for the desired reduction of the antibody production without being sufficient for blocking all antibodies present in the circulation of a patient at risk of sepsis or a patient suffering from sepsis.

1. Use of the gangliosides asialo-G_{M1} (AG_{M1}) and/or GM1 and of substances which simulate the carbohydrate moiety of these gangliosides with regard to binding to anti-AG_{M1} antibodies and/or anti-G_{M1} antibodies, for the preparation of an agent for binding or blocking anti-AG_{M1} antibodies and/or anti-G_{M1} antibodies which bind to natural killer cells (NKC), for administration to patients at risk of sepsis and patients suffering from sepsis, in whom such antibodies were detected, for the prevention, inhibition and treatment of sepsis.

2. Use of the gangliosides AG_{M1} and/or GM1 and of substances which simulate the carbohydrate moiety of these gangliosides with regard to binding to anti-AG_{M1} antibodies and/or anti-G_{M1} antibodies, for the preparation of agents for blocking antigen-presenting cells or for producing T-cell anergy in patients at risk of sepsis.

3. Use of the gangliosides AG_{M1} and/or G_{M1} and of substances which simulate the carbohydrate moiety of these gangliosides with regard to binding to anti-G_{M1} antibodies and/or anti-AG_{M1} antibodies, for the preparation of an affinity material for the extracorporeal removal of anti-AG_{M1} antibodies and/or anti-G_{M1} antibodies which bind to natural killer cells from the blood circulation of a patient at risk of sepsis or a patient suffering from sepsis.

4. Use according to claim 1, characterized in that the substances which simulate the carbohydrate moiety of these gangliosides with regard to binding to anti-AG_{M1} antibodies and/or anti-G_{M1} antibodies are characterized in that they are detectable with the aid of a screening method in which a substance to be tested is added to a serum having a high ganglioside antibody titre, the binding behaviour of the antibodies from the sample to the specific binding partners is then determined and the binding behaviour which is reduced compared with the substance-free sample is correlated with ganglioside simulation.

5. Use according to claim 2 for the preparation of an agent for blocking antigen-presenting cells or for producing T-cell anergy for administration by injection or for oral administration.

6. Agent for the prevention and treatment of sepsis, which, in addition to pharmaceutically acceptable excipients, contains active constituents which bind to AG_{M1} and/or G_{M1} antibodies in a manner such that the binding of these antibodies to natural killer cells (NKC) is completely or partly prevented.

7. Use according to claim 2, characterized in that the substances which simulate the carbohydrate moiety of these gangliosides with regard to binding to anti-AG_{M1} antibodies and/or anti-G_{M1} antibodies are characterized in that they are detectable with the aid of a screening method in which a substance to be tested is added to a serum having a high ganglioside antibody titre, the binding behaviour of the antibodies from the sample to the specific binding partners is then determined and the binding behaviour which is reduced compared with the substance-free sample is correlated with ganglioside simulation.

8. Use according to claim 3, characterized in that the substances which simulate the carbohydrate moiety of these gangliosides with regard to binding to anti-AG_{M1} antibodies and/or anti-G_{M1} antibodies are characterized in that they are detectable with the aid of a screening method in which a substance to be tested is added to a serum having a high ganglioside antibody titre, the binding behaviour of the antibodies from the sample to the specific binding partners is then determined and the binding behaviour which is reduced compared with the substance-free sample is correlated with ganglioside simulation.
